(19) United States
(12) Patent Application Publication
(10) Pub. No.: US 2018/0064644 A1
(43) Pub. Date: Mar. 8, 2018

(54) CHILDRENS COUGH AND SORE THROAT GRANULAR SYSTEMS

(71) Applicant: Summer Orton, Ainsworth, NE (US)

(72) Inventor: Summer Orton, Ainsworth, NE (US)

(21) Appl. No.: 15/257,903

(22) Filed: Sep. 6, 2016

(51) Int. Cl. A61K 9/00; A61K 47/44; A61K 31/045; A61K 36/00

(52) U.S. Cl. CPC A61K 9/0056; A61K 36/00; A61K 31/045; A61K 47/44

(57) ABSTRACT

A children's cough and sore throat granular system for effectively eliminating cough and cold symptoms in a safe and convenient way. Children's cough and sore throat granular system is a granular cough medicine for children and elderly consumers. It is a small sized cough and sore throat drops that give parents peace of mind while eliminating coughing in their young children.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of cold and flu medicine and more specifically relates to children's cough and sore throat granular systems.

### 2. Description of Related Art

A cough medicine or cough and cold medicine, also known as cough syrup or linctus when in syrup form, is a medicinal drug used in those with coughing and related conditions. Sometimes getting a young child to take medicine may be difficult. Thus the need for an alternative form of taking medicine.

Several attempts have been made to solve the above-mentioned problems such as those found in U.S. patent and U.S. Publication numbers: U.S. Pat. No. 4,551,329 to Joan Harris; U.S. Pat. No. 5,827,527 to Dana Leonard; and 2004/0170725 to Eric Begleiter. This art is representative of cold and flu medicine. However, none of the above inventions and patents, taken either singly or in combination, is seen to describe the invention as claimed.

Preferably, a cold and flu medicine should provide a convenient and safe alternative to traditional cold medicine and, yet would operate reliably and be manufactured at a modest expense. Thus, a need exists for a reliable children's cough and sore throat granular system to avoid the above-mentioned problems.

### BRIEF SUMMARY OF THE INVENTION

In view of the foregoing disadvantages inherent in the known cold and flu medicine art, the present invention provides a novel cold and flu medicine. The general purpose of the present invention, which will be described subsequently in greater detail is to provide a children's cough and sore throat granular system.

A children's cough and sore throat granular system is disclosed in a preferred embodiment comprising an edible medicated hard granule including an outer shell, a medicated center portion having at least one cough suppressing ingredient (which may be integral with outer shell), and a syrup base. The children's cough and sore throat granular system comprises the edible medicated hard granule. The edible medicated hard granule includes the outer shell and the medicated center. The edible medicated hard granule may comprise coloring. The coloring may be natural or artificial. The edible medicated hard granule may comprise at least one flavor.

The medicated center may comprise cough syrup or other suitable equivalent. The medicated center portion may comprise the at least one cough suppressing ingredient and the syrup base. The cough suppressing ingredient may comprise menthol or the like. The edible medicated hard granule may comprise an anti-sticking ingredient to avoid the edible medicated hard granules from sticking to each other when stored and during consumption. The anti-sticking ingredient may comprise carnauba wax. The edible medicated hard granule is designed for child oral consumption. The edible medicated hard granule gradually dissolves when orally consumed. The edible medicated hard granule provides a safe alternative to cough drops and cough syrup preventing overdoses and choking while consuming and motivate children to consume medicine when sick

The present invention holds significant improvements and serves as a children's cough and sore throat granular system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, children's cough and sore throat granular systems, constructed and operative according to the teachings of the present invention.

FIG. 1 shows a perspective view illustrating a children's cough and sore throat granular system comprising an edible medicated hard granule according to an embodiment of the present invention.

FIG. 2 is a perspective view illustrating the edible medicated hard granule according to an embodiment of the present invention of FIG. 1.

FIG. 3 is a perspective view illustrating the edible medicated hard granule according to an embodiment of the present invention.

FIG. 4 is a perspective view illustrating the edible medicated hard granule according to an embodiment of the present invention.

The various embodiments of the present invention will hereinafter be described in conjunction with the appended drawings, wherein like designations denote like elements.

### DETAILED DESCRIPTION

As discussed above, embodiments of the present invention relate to cold and flu medicine and more particularly to a children's cough and sore throat granular system as used to improve the safety and use of traditional cough syrup and cough drops.

Generally speaking, children's cough and sore throat granular systems may comprise granular cough medicine for children and elderly consumers. Choking is a serious hazard for young children. Often times, cough syrups contain ingredients with negative side effects. Small sized cough and sore throat drops give parents peace of mind while eliminating coughing in their young children. The present invention is designed to be orally consumed. The medicated granules don't dissolve quickly in the consumer's mouth. The granular may comprise similar ingredients as cough drops. The granules may also comprise an anti-sticking agent that prevents the granules from sticking together after becoming wet in a consumer's mouth. The medicated granule may comprise a syrup type base including brown rice syrup, glucose syrup or corn syrup. The granule may further comprise active ingredients such as menthol, essential oils and herbs.

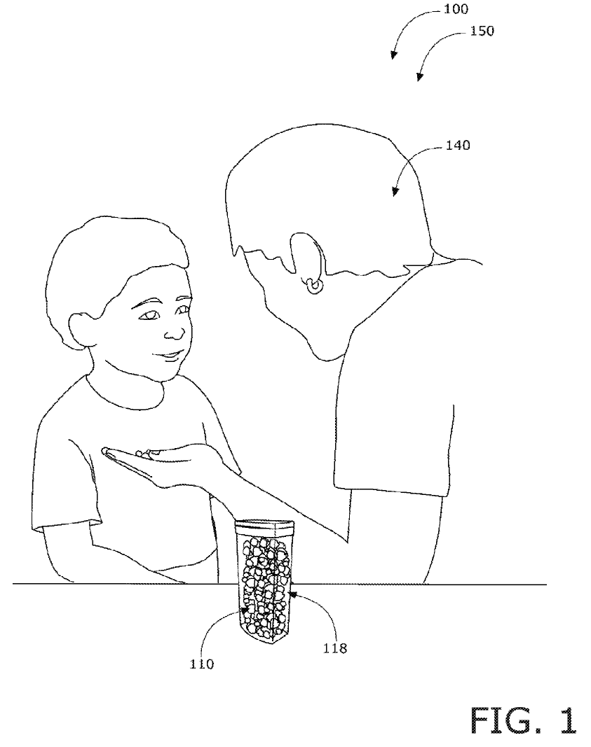

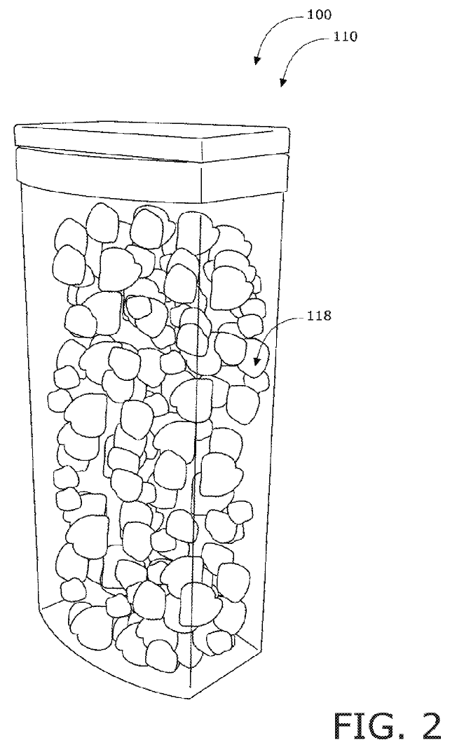

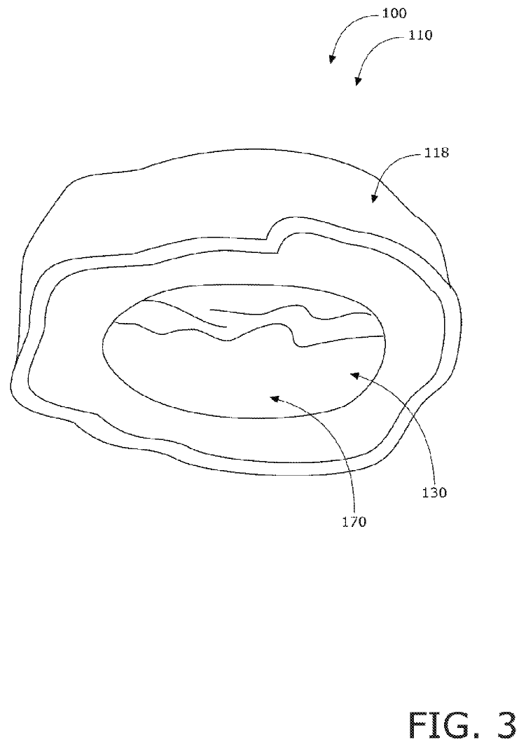

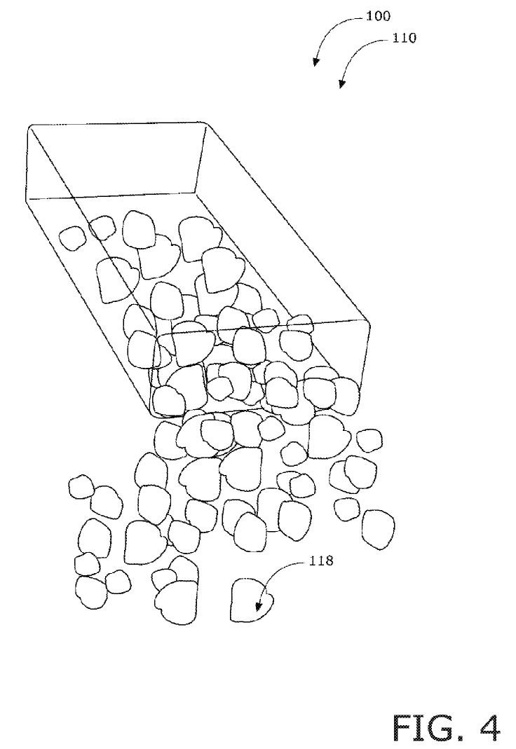

Referring to the drawings by numerals of reference there is shown in FIGS. 1-4, children's cough and sore throat granular system 100 according to an embodiment of the present invention. Children's cough and sore throat granular system 100 may edible medicated hard granule 110, which may comprise outer shell 118, medicated center portion 130 having at least one cough suppressing ingredient 160, and syrup base 170.

Children's cough and sore throat granular system 100 may comprise edible medicated hard granule 110. Edible medicated hard granule 110 includes outer shell 118 and medicated center 130. Medicated center portion 130 may comprise at least one cough suppressing 160 ingredient and syrup base 170. Edible medicated hard granule 110 provides a safe alternative to cough drops and cough syrup preventing overdoses and choking while consuming and motivates children to consume medicine when sick.

Edible medicated hard granule 110 is designed for child oral consumption. Edible medicated hard granule 110 is designed for oral consumption by elderly and those with disabilities. Edible medicated hard granule 110 may comprise at least one flavor. A medicated center 130 may comprise cough syrup. Edible medicated hard granule 110 may comprise an anti-sticking ingredient to avoid edible medicated hard granules 110 from sticking to each other when stored and during consumption. Anti-sticking ingredient may comprise carnauba wax. Cough suppressing ingredient 160 may comprise menthol. Cough suppressing ingredient 160 may comprise essential oils. Edible medicated hard granule 110 may comprise coloring. Coloring may be artificial or natural. Edible medicated hard granule 110 gradually dissolves when orally consumed. Children's cough and sore throat granular system 100 may be manufactured and provided for sale in a wide variety of sizes and shapes for a wide assortment of applications.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A children's cough and sore throat granular system comprising:
an edible medicated hard granule including;
an outer shell;
a medicated center portion having;
at least one cough suppressing ingredient; and
a syrup base;
wherein said children's cough and sore throat granular system comprises said edible medicated hard granule;
wherein said edible medicated hard granule includes said outer shell and said medicated center;
wherein said medicated center portion comprises said at least one cough suppressing ingredient and said syrup base; and
wherein said edible medicated hard granule provides a safe alternative to cough drops and cough syrup preventing overdoses and choking while consuming and motivate children to consume medicine when sick.

2. The children's cough and sore throat granular system of claim 1 wherein said edible medicated hard granule is designed for child oral consumption.

3. The children's cough and sore throat granular system of claim 1 wherein said edible medicated hard granule is designed for oral consumption by elderly and those with disabilities.

4. The children's cough and sore throat granular system of claim 1 wherein said edible medicated hard granule comprises at least one flavor.

5. The children's cough and sore throat granular system of claim 1 wherein said a medicated center comprises cough syrup.

6. The children's cough and sore throat granular system of claim 1 wherein said edible medicated hard granule comprises an anti-sticking ingredient to avoid said edible medicated hard granules from sticking to each other when stored and during consumption.

7. The children's cough and sore throat granular system of claim 1 wherein said anti-sticking ingredient comprises carnauba wax.

8. The children's cough and sore throat granular system of claim 1 wherein said cough suppressing ingredient comprises menthol.

9. The children's cough and sore throat granular system of claim 1 wherein said cough suppressing ingredient comprises essential oils.

10. The children's cough and sore throat granular system of claim 1 wherein said edible medicated hard granule comprises coloring.

11. The children's cough and sore throat granular system of claim 10 wherein said coloring is artificial.

12. The children's cough and sore throat granular system of claim 10 wherein said coloring is natural.

13. The children's cough and sore throat granular system of claim 1 wherein said edible medicated hard granule gradually dissolves when orally consumed.

14. A children's cough and sore throat granular system comprising:
an edible medicated hard granule including;
an outer shell;
a medicated center portion having;
at least one cough suppressing ingredient; and
a syrup base;
wherein said children's cough and sore throat granular system comprises said edible medicated hard granule;
wherein said edible medicated hard granule includes said outer shell and said medicated center;
wherein said edible medicated hard granule comprises coloring;
wherein said coloring is natural;
wherein said edible medicated hard granule comprises at least one flavor;
wherein said a medicated center comprises cough syrup;
wherein said medicated center portion comprises said at least one cough suppressing ingredient and said syrup base;
wherein said cough suppressing ingredient comprises menthol;
wherein said edible medicated hard granule comprises an anti-sticking ingredient to avoid said edible medicated hard granules from sticking to each other when stored and during consumption;
wherein said anti-sticking ingredient comprises carnauba wax;
wherein said edible medicated hard granule is designed for child oral consumption;
wherein said edible medicated hard granule gradually dissolves when orally consumed; and
wherein said edible medicated hard granule provides a safe alternative to cough drops and cough syrup preventing overdoses and choking while consuming and motivate children to consume medicine when sick.
